(19) United States
(12) Patent Application Publication
(10) Pub. No.: US 2026/0272402 A1
(43) Pub. Date: Sep. 17, 2026

(54) Method For Quantifying Breast Density Or Glandular Tissue Components Using Ultrasound Image

(71) Applicant: BEAMWORKS INC., Daegu (KR)

(72) Inventors: Ho Kyung SHIN, Daegu (KR); Won Hwa KIM, Daegu (KR); Jaeil KIM, Daegu (KR)

(73) Assignee: BEAMWORKS INC., Daegu (KR)

(21) Appl. No.: 19/167,250

(22) PCT Filed: Jan. 15, 2024
(86) PCT No.: PCT/KR2024/000676

§ 371 Date: Sep. 19, 2025

(30) Foreign Application Priority Data
Mar. 30, 2023 (KR) 10-2023-0041922

(51) Int. Cl. A61B 8/08; A61B 8/00; G06T 7/00; G06T 7/11

(52) U.S. Cl. CPC A61B 8/0825; A61B 8/469; A61B 8/5223; G06T 7/0012; G06T 7/11; G06T 2207/10132; G06T 2207/20072; G06T 2207/20084; G06T 2207/30068

(57) ABSTRACT

Disclosed is a method for quantifying breast density or glandular tissue components using an ultrasound image, which is performed by a computing device. The method may include: acquiring an ultrasound image; identifying a breast region in the ultrasound image; identifying a fibrous glandular tissue region in the ultrasound image; and quantifying a density of a breast by analyzing the identified breast region and fibrous glandular tissue region.

### TECHNICAL FIELD

The present disclosure relates to a method for quantifying breast density or glandular tissue components using an ultrasound image.

### BACKGROUND ART

Breast cancer has a large difference in survival rate depending on the time of treatment. Adult women are periodically undergoing mammography screening for early diagnosis of breast cancer. Mammography is an X-ray examination that shows soft tissue images of the breast. Breast density varies by race, and in Korean, about 54% or more present as dense breasts. Dense breasts are less prone to suspicious lesions, which affects diagnostic accuracy. Breast ultrasound is most commonly used for cancer screening of dense breasts. Breast density and density of glandular tissue components have been known to be associated with the development of breast cancer, and therefore, evaluation of the readout of breast density in breast ultrasound diagnosis is performed together. In mammography, dense breast has a limitation in that it is difficult to distinguish malignant breast tissue, but breast ultrasound can distinguish mammary tissue components. However, in breast ultrasound diagnosis, the breast density and the density of mammary gland tissue components are graded by the subjective judgment of the examiner, and there is a problem that they are not quantified. Korean Patent Unexamined Publication No. 10-2023-0025555 (Feb. 22, 2023) discloses systems and methods for assessing a risk of breast cancer.

### DISCLOSURE

### Technical Problem

The present disclosure has been made in an effort to provide a method for quantifying breast density or glandular tissue components by analyzing an ultrasound image and utilizing the quantified breast density or glandular tissue components as an objective indicator in cancer screening.

Meanwhile, a technical object to be achieved by the present disclosure is not limited to the above-mentioned technical object, and various technical objects can be included within the scope which is obvious to those skilled in the art from contents to be described below.

### Technical Solution

In order to achieve the above-described object, disclosed is a method for quantifying breast density or glandular tissue components using an ultrasound image, which is performed by a computing device according to an embodiment of the present disclosure. The method may include: acquiring an ultrasound image; identifying a breast region in the ultrasound image; identifying a fibrous glandular tissue region in the ultrasound image; and quantifying a density of a breast by analyzing the identified breast region and fibrous glandular tissue region.

In an embodiment, the identifying of the breast region in the ultrasound image may include identifying the breast region by inputting the ultrasound image into a deep learning segmentation model.

In an embodiment, the identifying of the fibrous glandular tissue region in the ultrasound image may include distinguishing and identifying the breast region and the fibrous glandular tissue region jointly by inputting the ultrasound image into the deep learning segmentation model.

In an embodiment, the quantifying of the density of the breast by analyzing the identified breast region and fibrous glandular tissue region may include calculating a ratio of the fibrous glandular tissue region to a size of the identified breast region, and quantifying a density of a breast tissue based on the calculated ratio.

In an embodiment, the quantifying of the density of the breast tissue may include additionally quantifying the density of the breast tissue based on a mapping between the calculated ratio and a predefined breast density grade.

In an embodiment, the predefined breast density grade may include an ACR BI-RADS Tissue Composition grade.

In an embodiment, the method may further include identifying a glandular tissue component region within the identified fibrous glandular tissue region.

In an embodiment, the identifying of the glandular tissue component region within the identified fibrous glandular tissue region may include applying histogram equalization to the fibrous glandular tissue region, and detecting the glandular tissue component region in the fibrous glandular tissue region based on a statistical threshold.

In an embodiment, the method may further include performing an operation of quantifying glandular tissue components by analyzing the identified fibrous glandular tissue region and the identified glandular tissue component region.

In an embodiment, the performing of the operation of quantifying the glandular tissue components by analyzing the identified fibrous glandular tissue region and the identified glandular tissue component region may include calculating a ratio of a size of the glandular tissue component region to a size of the fibrous glandular tissue region, and quantifying the glandular tissue components based on the calculated ratio.

In an embodiment, the quantifying of the glandular tissue components may include additionally quantifying the glandular tissue components based on a mapping between the calculated ratio and a predefined glandular tissue component grade.

In an embodiment, the predefined glandular tissue component grade may include a Glandular Tissue Composition (GTC) grade.

In order to achieve the above-described object, disclosed is a computer program stored in a computer readable storage medium according to an embodiment of the present disclosure. When the computer program is executed by one or more processors, the computer program allows the one or more processors to perform the following operations for quantifying breast density and glandular tissue components using an ultrasound image, and the operations may include: an operation of acquiring an ultrasound image; an operation of identifying a breast region in the ultrasound image; an operation of identifying a fibrous glandular tissue region in the ultrasound image; and an operation of quantifying a density of a breast by analyzing the identified breast region and fibrous glandular tissue region.

In an embodiment, the operation of identifying the breast region in the ultrasound image may include an operation of identifying the breast region by inputting the ultrasound image into a deep learning segmentation model.

In an embodiment, the operation of identifying the fibrous glandular tissue region in the ultrasound image may include an operation of distinguishing and identifying the breast region and the fibrous glandular tissue region jointly by inputting the ultrasound image into the deep learning segmentation model.

In an embodiment, the operation of quantifying the density of the breast by analyzing the identified breast region and fibrous glandular tissue region may include an operation of calculating a ratio of the fibrous glandular tissue region to a size of the identified breast region, and an operation of quantifying the density of the breast tissue based on the calculated ratio.

In an embodiment, the operation of quantifying the density of the breast tissue may include an operation of additionally quantifying the density of the breast tissue based on a mapping between the calculated ratio and a predefined breast density grade. In an embodiment, the operations may further include an operation of identifying a glandular tissue component region within the identified fibrous glandular tissue region.

In an embodiment, the operation of identifying the glandular tissue component region within the identified fibrous glandular tissue region may include an operation of applying histogram equalization to the fibrous glandular tissue region, and an operation of detecting the glandular tissue component region in the fibrous glandular tissue region based on a statistical threshold.

In an embodiment, the operations may further include an operation of performing an operation of quantifying glandular tissue components by analyzing the identified fibrous glandular tissue region and the identified glandular tissue component region.

In an embodiment, the operation of performing the operation of quantifying the glandular tissue components by analyzing the identified fibrous glandular tissue region and the identified glandular tissue component region may include an operation of calculating a ratio of a size of the glandular tissue component region to a size of the fibrous glandular tissue region, and an operation of quantifying the glandular tissue components based on the calculated ratio.

In order to achieve the above-described object, disclosed is a computing device according to an embodiment of the present disclosure. The device may include: at least one processor; and a memory, and the at least one processor may be configured to acquire an ultrasound image, identify a breast region in the ultrasound image, identify a fibrous glandular tissue region in the ultrasound image, and quantify a density of a breast by analyzing the identified breast region and fibrous glandular tissue region.

In an embodiment, the at least one processor may be configured to identify the breast region by inputting the ultrasound image into a deep learning segmentation model.

In an embodiment, the at least one processor may be configured to distinguish and identify the breast region and the fibrous glandular tissue region jointly by inputting the ultrasound image into the deep learning segmentation model.

In an embodiment, the at least one processor may be configured to calculate a ratio of the fibrous glandular tissue region to a size of the identified breast region, and quantify a density of a breast tissue based on the calculated ratio.

In an embodiment, the at least one processor may be configured to identify the glandular tissue component region within the identified fibrous glandular tissue region. In an embodiment, the at least one processor may be configured to apply histogram equalization to the fibrous glandular tissue region, and detect the glandular tissue component region in the fibrous glandular tissue region based on a statistical threshold.

In an embodiment, the at least one processor may be configured to perform the operation of quantifying the glandular tissue components by analyzing the identified fibrous glandular tissue region and the identified glandular tissue component region.

### Advantageous Effects

According to the present disclosure, breast density and glandular tissue components can be quantified by analyzing an ultrasound image and the quantified breast density or glandular tissue components can be utilized as an objective indicator in cancer screening. For example, according to the present disclosure, it is possible to provide objective, quantitative, accurate, and reproducible screening results by minimizing the inter-observer variation between the radiologists and the intra-observer variations of the reading physicians themselves in diagnosing breast cancer by quantifying the breast density or glandular tissue components using only an “ultrasonic image” and providing the quantified breast density or glandular tissue components.

In addition, the present disclosure can provide objective, quantitative, accurate, and reproducible screening results by analyzing breast ultrasound images and classifying and quantifying glandular tissue components.

In addition, the present disclosure can overcome a limitation of a technique that utilize mammography (i.e., a limitation that dense breasts are difficult to distinguish malignant breast tissue in mammography).

Further, the present disclosure can provide quantified indicators for breast density and glandular tissue components that can be used in screening ultrasound applications, and provide an automatic measurement method for the indicators.

Meanwhile, the effects of the present disclosure are not limited to the above-mentioned effects, and various effects can be included within the scope which is obvious to those skilled in the art from contents to be described below.

### DESCRIPTION OF DRAWINGS

FIG. 1 is a block diagram of a computing device for quantifying breast density or glandular tissue components using an ultrasound image according to an embodiment of the present disclosure.

FIG. 2 is a conceptual diagram illustrating a neural network according to an embodiment of the present disclosure.

FIG. 3 is a flowchart of a method for quantifying breast density or glandular tissue components using an ultrasound image according to an embodiment of the present disclosure.

FIG. 4 is a diagram for describing an operation of identifying a breast region and a fibrous glandular tissue region in an ultrasound image according to an embodiment of the present disclosure.

FIG. 5 is a diagram for describing an operation of identifying a glandular tissue component region within the fibrous glandular tissue region identified in the ultrasound image according to an embodiment of the present disclosure.

FIG. 6 is a simple and normal schematic diagram of an exemplary computing environment in which the embodiments of the present disclosure may be implemented.

### BEST MODEL

Various exemplary embodiments will now be described with reference to drawings. In the present specification, various descriptions are presented to provide appreciation of the present disclosure. However, it is apparent that the exemplary embodiments can be executed without the specific description.

“Component”, “module”, “system”, and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “at least one of A or B” should be interpreted to mean “a case including only A”, “a case including only B”, and “a case in which A and B are combined”.

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

In the present disclosure, a network function and an artificial neural network and a neural network may be interchangeably used.

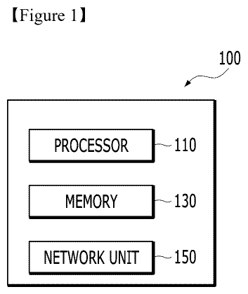

FIG. 1 is a block diagram of a computing device for quantifying breast density or glandular tissue components using an ultrasound image according to an embodiment of the present disclosure.

A configuration of the computing device 100 illustrated in FIG. 1 is only an example shown through simplification. In an exemplary embodiment of the present disclosure, the computing device 100 may include other components for performing a computing environment of the computing device 100 and only some of the disclosed components may constitute the computing device 100.

The computing device 100 may include a processor 110, a memory 130, and a network unit 150.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in an exemplary embodiment of the present disclosure, processors of a plurality of computing devices may be used together to process the learning of the network function and the data classification using the network function. Further, the computer program executed in the computing device according to an exemplary embodiment of the present disclosure may be a CPU, GPGPU, or TPU executable program.

According to an exemplary embodiment of the present disclosure, the memory 130 may store any type of information generated or determined by the processor 110 and any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit 150 according to an exemplary embodiment of the present disclosure may use various wired communication systems such as a Public Switched Telephone Network (PSTN), xDSL (x Digital Subscriber Line), RADSL (Rate Adaptive DSL), MDSL (Multi Rate DSL), VDSL (Very High Speed DSL), UADSL (Universal Asymmetric DSL), HDSL (High Bit Rate DSL), and a Local Area Network (LAN).

Further, the network unit 150 presented in this specification may use various wireless communication systems such as code division multi access (CDMA), time division multi access (TDMA), frequency division multi access (FDMA), orthogonal frequency division multi access (OFDMA), single carrier-FDMA (SC-FDMA), and other systems.

In the present disclosure, the network unit 150 may be configured regardless of communication modes such as wired and wireless modes and constituted by various communication networks including a local area network (LAN), a personal area network (PAN), a wide area network (WAN), and the like. Further, the network may be known World Wide Web (WWW) and may adopt a wireless transmission technology used for short-distance communication, such as infrared data association (IrDA) or Bluetooth.

The techniques described in this specification may also be used in other networks in addition to the aforementioned networks.

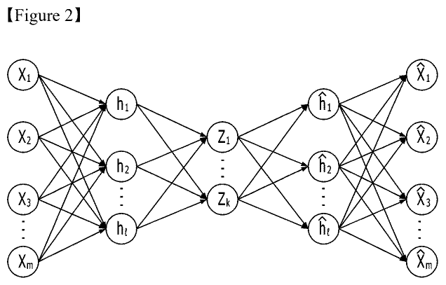

FIG. 2 is a schematic diagram illustrating a network function according to an exemplary embodiment of the present disclosure.

Throughout the present specification, a computation model, the neural network, a network function, and the neural network may be used as the same meaning. The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the link may relatively form the relationship between an input node and an output node. Concepts of the input node and the output node are relative and a predetermined node which has the output node relationship with respect to one node may have the input node relationship in the relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and the weight is variable by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights granted to the respective links in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, a definition of the layer is predetermined for description and the order of the layer in the neural network may be defined by a method different from the aforementioned method. For example, the layers of the nodes may be defined by the distance from a final output node.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean nodes constituting the neural network other than the initial input node and the final output node.

In the neural network according to an exemplary embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another exemplary embodiment of the present disclosure, the number of nodes of the input layer may be smaller than the number of nodes of the output layer, and the neural network may be a neural network of a type in which the number of nodes decreases from the input layer to the hidden layer. Further, in the neural network according to yet another exemplary embodiment of the present disclosure, the number of nodes of the input layer may be larger than the number of nodes of the output layer, and the neural network may be a neural network of a type in which the number of nodes increases from the input layer to the hidden layer. The neural network according to still yet another exemplary embodiment of the present disclosure may be a neural network of a type in which the neural networks are combined.

A deep neural network (DNN) may refer to a neural network that includes a plurality of hidden layers in addition to the input and output layers. When the deep neural network is used, the latent structures of data may be determined. That is, latent structures of photos, text, video, voice, and music (e.g., what objects are in the photo, what the content and feelings of the text are, what the content and feelings of the voice are) may be determined. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siam network, a Generative Adversarial Network (GAN), and the like. The description of the deep neural network described above is just an example and the present disclosure is not limited thereto.

In an exemplary embodiment of the present disclosure, the network function may include the auto encoder. The auto encoder may be a kind of artificial neural network for outputting output data similar to input data. The auto encoder may include at least one hidden layer and odd hidden layers may be disposed between the input and output layers. The number of nodes in each layer may be reduced from the number of nodes in the input layer to an intermediate layer called a bottleneck layer (encoding), and then expanded symmetrical to reduction to the output layer (symmetrical to the input layer) in the bottleneck layer. The auto encoder may perform non-linear dimensional reduction. The number of input and output layers may correspond to a dimension after preprocessing the input data. The auto encoder structure may have a structure in which the number of nodes in the hidden layer included in the encoder decreases as a distance from the input layer increases. When the number of nodes in the bottleneck layer (a layer having a smallest number of nodes positioned between an encoder and a decoder) is too small, a sufficient amount of information may not be delivered, and as a result, the number of nodes in the bottleneck layer may be maintained to be a specific number or more (e.g., half of the input layers or more).

The neural network may be learned in at least one scheme of supervised learning, unsupervised learning, semi supervised learning, or reinforcement learning. The learning of the neural network may be a process in which the neural network applies knowledge for performing a specific operation to the neural network.

The neural network may be learned in a direction to minimize errors of an output. The learning of the neural network is a process of repeatedly inputting learning data into the neural network and calculating the output of the neural network for the learning data and the error of a target and back-propagating the errors of the neural network from the output layer of the neural network toward the input layer in a direction to reduce the errors to update the weight of each node of the neural network. In the case of the supervised learning, the learning data labeled with a correct answer is used for each learning data (i.e., the labeled learning data) and in the case of the unsupervised learning, the correct answer may not be labeled in each learning data. That is, for example, the learning data in the case of the supervised learning related to the data classification may be data in which category is labeled in each learning data. The labeled learning data is input to the neural network, and the error may be calculated by comparing the output (category) of the neural network with the label of the learning data. As another example, in the case of the unsupervised learning related to the data classification, the learning data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network ensures a certain level of performance quickly by using a high learning rate, thereby increasing efficiency and uses a low learning rate in a latter stage of the learning, thereby increasing accuracy.

In learning of the neural network, the learning data may be generally a subset of actual data (i.e., data to be processed using the learned neural network), and as a result, there may be a learning cycle in which errors for the learning data decrease, but the errors for the actual data increase. Overfitting is a phenomenon in which the errors for the actual data increase due to excessive learning of the learning data. For example, a phenomenon in which the neural network that learns a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the learning data, regularization, dropout of omitting a part of the node of the network in the process of learning, utilization of a batch normalization layer, etc., may be applied.

According to AN embodiment of the present disclosure, breast density and density of glandular tissue components are associated with breast cancer development. In dense breasts, breast density may be confirmed through mammography, but breast tissue separation is difficult, and calculation of a glandular tissue component ratio is limited. In order to solve this problem, the present disclosure can identify each of a breast region and a fibrous glandular tissue region using only an “ultrasonic image”, and analyze the identified breast region and fibrous glandular tissue region to quantify the breast density or glandular tissue components. Further, the present disclosure can provide objective, quantitative, accurate, and reproducible screening results by minimizing the inter-observer variation between the reading physicians and the intra-observer variations of the reading physicians themselves in diagnosing breast cancer by quantifying and providing the breast density or glandular tissue components using only an “ultrasonic image”.

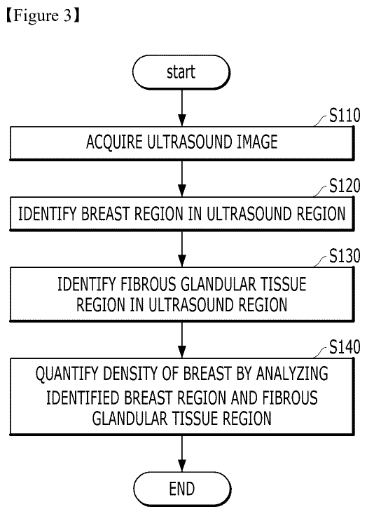

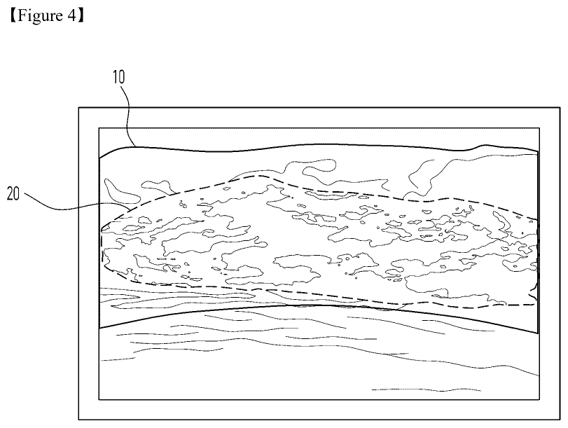

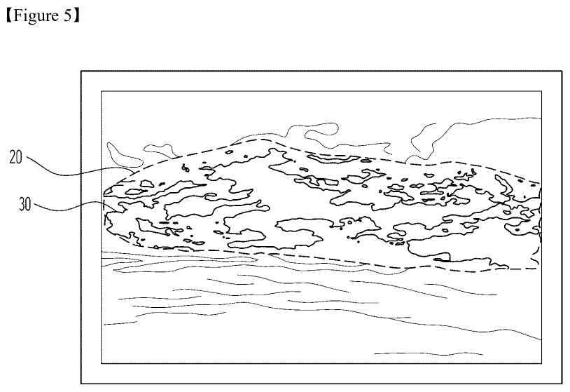

FIG. 3 is a flowchart of a method for quantifying breast density or glandular tissue components using an ultrasound image according to an embodiment of the present disclosure, FIG. 4 is a diagram for describing an operation of identifying a breast region and a fibrous glandular tissue region in an ultrasound image according to an embodiment of the present disclosure, and FIG. 5 is a diagram for describing an operation of identifying a glandular tissue component region within the fibrous glandular tissue region identified in the ultrasound image according to an embodiment of the present disclosure.

Referring to FIG. 3, a method for quantifying breast density or glandular tissue components using an ultrasound image according to an embodiment of the present disclosure may include a step S110 of obtaining an ultrasound image, a step S120 of identifying a breast region in the ultrasound image, a step S130 of identifying a fibrous glandular tissue region in the ultrasound image, and a step S140 of quantifying a density of the breast by analyzing the identified breast region and fibrous glandular tissue region. Further, the method for quantifying breast density or glandular tissue components using an ultrasound image according to an embodiment of the present disclosure may be performed by the computing device 100.

According to an embodiment of the present disclosure, the computing device 100 may obtain an ultrasound image (S110). The ultrasound image may be an ultrasound image acquired by an ultrasound probe which is equipment that receives an echo reflected after ultrasound inspection equipment generates and transmits an ultrasound wave. Further, the ultrasound image may include a medical image acquired by a freeze mode of the probe is activated. As an example, the computing device 100 may scan a breast and obtain an ultrasound image by using ultrasound equipment (e.g., an ultrasound probe, etc.).

According to an embodiment of the present disclosure, the computing device 100 may identify a breast region in the ultrasound image (S120). For example, the computing device 100 may identify the breast region 10 by inputting the ultrasound image into a deep learning segmentation model. For reference, a breast is composed of epithelial tissue, connective tissue, and fat. Referring illustratively to FIG. 4, the computing device 100 may identify the breast region 10 in the ultrasound image in order to more accurately identify breast density or glandular tissue components. As an example, in order to identify the breast region 10, various deep learning segmentation models including U-NET, MASK R-CNN, DeepLap, PSPNet, Fully Convolutional Network (FCN), SegNet, ENet, etc., may be applied, but are not limited thereto, and an algorithm which is previously developed or developed afterward may be applied. As an example, the computing device 100 may represent the breast region 10 in the form of a line or dotted line using a first color (e.g., blue) and provide the breast region 10 through a user interface as illustrated in FIG. 4.

According to an embodiment of the present disclosure, the computing device 100 may identify the fibrous glandular tissue region in the ultrasound image (S130). For example, the computing device 100 may distinguish and identify the breast region 10 and the fibrous glandular tissue region 20 jointly by inputting the ultrasound image into the deep learning segmentation model. Referring exemplarily to FIG. 4, the computing device 100 may distinguish and identify the breast region 10 and the fibrous glandular tissue region 20 jointly by inputting the ultrasound image into the deep learning segmentation model. As an example, in order to identify the breast region 10 and the fibrous glandular tissue region 20 jointly, various deep learning segmentation models including U-NET, MASK R-CNN, DeepLap, PSPNet, Fully Convolutional Network (FCN), SegNet, ENet, etc., may be applied, but are not limited thereto, and an algorithm which is previously developed or developed afterward may be applied. As an example, the computing device 100 may represent the fibrous glandular tissue region 20 in the form of a line or dotted line using a second color (e.g., yellow) and provide the breast region 20 through a user interface as illustrated in FIG. 4. The computing device 100 distinguishes the breast region 10 using the first color (e.g., blue) and distinguishes and provides the fibrous glandular tissue region 20 using the second color (e. g., yellow) to be identifiable together, so that a user (e.g., a physician) may more clearly determine the identified region using the deep learning segmentation model.

According to an embodiment, the computing device 100 may identify the glandular tissue component region within the identified fibrous glandular tissue region. For example, the computing device 100 may input the ultrasound image into a rule-based model or the deep learning segmentation model to identify a glandular tissue component region 30 within the identified fibrous glandular tissue region 20. As an example, the computing device 100 may apply histogram equalization to the fibrous glandular tissue region.

For reference, histogram smoothing refers to a processing method for evenly distributing contrast values when there is an image in which the distribution of contrast values is biased to one side. In addition, the computing device 100 may detect the glandular tissue region component region 30 in the fibrous glandular tissue region 20 based on a statistical threshold. Exemplarily referring to FIG. 5, since the glandular tissue component in the glandular ultrasound image has a contrast distribution that is distinct from other tissues of the fibrous glandular tissue, the computing device 100 may detect the glandular tissue component region 30 in the fibrous glandular tissue region 20 based on the statistical threshold after applying the histogram equalization to the fibrous glandular tissue region. The computing device 100 may also represent the detected glandular tissue component region 30 in the form of a line or dotted line using a third color (e.g., red) and provide the glandular tissue component region 30 through the user interface.

According to an embodiment of the present disclosure, the computing device 100 may quantify the density of the breast by analyzing the identified breast region 10 and the fibrous mammary tissue region 20 (S140). As an example, the density of the breast refers to a qualitative or quantitative measurement of a proportion of related tissues excluding fat in the entire breast region, and a qualitative classification may distinguish the breast density according to a BI-RADS protocol. Exemplarily referring to FIG. 4, the computing device 100 may calculate a ratio of the fibrous glandular tissue region 20 to a size of the identified breast region 10. The computing device 100 may calculate the ratio of the fibrous glandular tissue region 20 to the size of the identified breast region 10, and represent the ratio as a percentage. Further, the computing device 100 may quantify a density of the breast tissue based on the calculated ratio. For example, the computing device 100 may calculate the ratio of the fibrous glandular tissue region 20 identified in step S130 to the size of the breast region 10 identified in step S120, and quantify the density of the breast tissue based on the ratio.

Additionally, the computing device 100 may additionally quantify the density of the breast tissue based on a mapping between the calculated ratio and a predefined breast density grade. In other words, the computing device 100 may primarily quantify the density of breast tissue by calculating the ratio of the fibrous glandular tissue region 20 to the size of the identified breast region 10, and further quantify the density of the breast tissue (quantification with grade information) based on the mapping between the calculated ratio and the predefined breast density grade. Here, the predefined breast density grade may include an ACR BI-RADS Tissue Composition grade. For reference, the ACR BI-RADS Tissue Composition grade is defined as a background echo in terms of the balance between FGT and fat and may be classified into three categories: homogeneous-fat, homogeneous-fibroglandular, and heterogeneous.

According to an embodiment, the computing device 100 may quantify the glandular tissue components (glandular tissue component density) by analyzing the identified fibrous glandular tissue region 20 and the identified glandular tissue component region 30. For example, the computing device 100 may calculate a ratio of the size of the glandular tissue component region to the size of the fibrous glandular tissue region. Further, the computing device 100 may quantify the glandular tissue components (glandular tissue component density) based on the calculated ratio.

Additionally, the computing device 100 may additionally quantify (quantify with grade information) the glandular tissue components (glandular tissue component density) based on a mapping between the calculated ratio and a predefined glandular tissue component grade. Here, the predefined glandular tissue component grade may include a Glandular Tissue Composition (GTC) grade. For reference, evaluation of the GTC means a ratio of a gray region indicating the glandular tissue to a white region indicating a fibrotic epilepsy in a fibroglandular tissue (FGT) surrounded by subcutaneous and glandular olfactoriae fat in a breast ultrasound. At this time, a fat lobule of the FGT distinguished from the tissue is not included in the GTC evaluation. The glandular tissue composition (GTC) grade may be normally classified into minimum (less than 25% of FGT), mild (25 to 49% of FGT), moderate (50 to 74% of FGT), or marked (75% or more of FGT) for each woman based on both breast ultrasound images, and written as abbreviations of P1, P2, P3, and P4 in a reading text. The GTC may reclassify the minimal or mild GTC as “low” and the moderate or marked GTC as “high” based on GTCs constituting 50% of the breast FGT.

In the above description, steps S110 to S140 may further be divided into additional steps, or combined as fewer steps according to an implementation example of the present disclosure. Further, some steps may also be omitted as necessary, and an order between the steps may be changed.

Disclosed is a computer readable medium storing the data structure according to an exemplary embodiment of the present disclosure.

The data structure may refer to the organization, management, and storage of data that enables efficient access to and modification of data. The data structure may refer to the organization of data for solving a specific problem (e.g., data search, data storage, data modification in the shortest time). The data structures may be defined as physical or logical relationships between data elements, designed to support specific data processing functions. The logical relationship between data elements may include a connection relationship between data elements that the user defines. The physical relationship between data elements may include an actual relationship between data elements physically stored on a computer-readable storage medium (e.g., persistent storage device). The data structure may specifically include a set of data, a relationship between the data, a function which may be applied to the data, or instructions. Through an effectively designed data structure, a computing device can perform operations while using the resources of the computing device to a minimum. Specifically, the computing device can increase the efficiency of operation, read, insert, delete, compare, exchange, and search through the effectively designed data structure.

The data structure may be divided into a linear data structure and a non-linear data structure according to the type of data structure. The linear data structure may be a structure in which only one data is connected after one data. The linear data structure may include a list, a stack, a queue, and a deque. The list may mean a series of data sets in which an order exists internally. The list may include a linked list. The linked list may be a data structure in which data is connected in a scheme in which each data is linked in a row with a pointer. In the linked list, the pointer may include link information with next or previous data. The linked list may be represented as a single linked list, a double linked list, or a circular linked list depending on the type. The stack may be a data listing structure with limited access to data. The stack may be a linear data structure that may process (e.g., insert or delete) data at only one end of the data structure. The data stored in the stack may be a data structure (LIFO-Last in First Out) in which the data is input last and output first. The queue is a data listing structure that may access data limitedly and unlike a stack, the queue may be a data structure (FIFO-First in First Out) in which late stored data is output late. The deque may be a data structure capable of processing data at both ends of the data structure.

The non-linear data structure may be a structure in which a plurality of data are connected after one data. The non-linear data structure may include a graph data structure. The graph data structure may be defined as a vertex and an edge, and the edge may include a line connecting two different vertices. The graph data structure may include a tree data structure. The tree data structure may be a data structure in which there is one path connecting two different vertices among a plurality of vertices included in the tree. That is, the tree data structure may be a data structure that does not form a loop in the graph data structure.

Throughout the present specification, a computation model, the neural network, a network function, and the neural network may be used as the same meaning. Hereinafter, the computation model, the neural network, the network function, and the neural network will be integrated and described as the neural network. The data structure may include the neural network. In addition, the data structures, including the neural network, may be stored in a computer readable medium. The data structure including the neural network may also include data preprocessed for processing by the neural network, data input to the neural network, weights of the neural network, hyper parameters of the neural network, data obtained from the neural network, an active function associated with each node or layer of the neural network, and a loss function for learning the neural network. The data structure including the neural network may include predetermined components of the components disclosed above. In other words, the data structure including the neural network may include all of data preprocessed for processing by the neural network, data input to the neural network, weights of the neural network, hyper parameters of the neural network, data obtained from the neural network, an active function associated with each node or layer of the neural network, and a loss function for learning the neural network or a combination thereof. In addition to the above-described configurations, the data structure including the neural network may include predetermined other information that determines the characteristics of the neural network. In addition, the data structure may include all types of data used or generated in the calculation process of the neural network, and is not limited to the above. The computer readable medium may include a computer readable recording medium and/or a computer readable transmission medium. The neural network may be generally constituted by an aggregate of calculation units which are mutually connected to each other, which may be called nodes. The nodes may also be called neurons. The neural network is configured to include one or more nodes.

The data structure may include data input into the neural network. The data structure including the data input into the neural network may be stored in the computer readable medium. The data input to the neural network may include learning data input in a neural network learning process and/or input data input to a neural network in which learning is completed. The data input to the neural network may include preprocessed data and/or data to be preprocessed. The preprocessing may include a data processing process for inputting data into the neural network. Therefore, the data structure may include data to be preprocessed and data generated by preprocessing. The data structure is just an example and the present disclosure is not limited thereto.

The data structure may include the weight of the neural network (in the present disclosure, the weight and the parameter may be used as the same meaning). In addition, the data structures, including the weight of the neural network, may be stored in the computer readable medium. The neural network may include a plurality of weights. The weight may be variable and the weight is variable by a user or an algorithm in order for the neural network to perform a desired function. For example, when one or more input nodes are mutually connected to one output node by the respective links, the output node may determine a data value output from an output node based on values input in the input nodes connected with the output node and the weights set in the links corresponding to the respective input nodes. The data structure is just an example and the present disclosure is not limited thereto.

As a non-limiting example, the weight may include a weight which varies in the neural network learning process and/or a weight in which neural network learning is completed. The weight which varies in the neural network learning process may include a weight at a time when a learning cycle starts and/or a weight that varies during the learning cycle. The weight in which the neural network learning is completed may include a weight in which the learning cycle is completed. Accordingly, the data structure including the weight of the neural network may include a data structure including the weight which varies in the neural network learning process and/or the weight in which neural network learning is completed. Accordingly, the above-described weight and/or a combination of each weight are included in a data structure including a weight of a neural network. The data structure is just an example and the present disclosure is not limited thereto.

The data structure including the weight of the neural network may be stored in the computer-readable storage medium (e.g., memory, hard disk) after a serialization process. Serialization may be a process of storing data structures on the same or different computing devices and later reconfiguring the data structure and converting the data structure to a form that may be used. The computing device may serialize the data structure to send and receive data over the network. The data structure including the weight of the serialized neural network may be reconfigured in the same computing device or another computing device through deserialization. The data structure including the weight of the neural network is not limited to the serialization. Furthermore, the data structure including the weight of the neural network may include a data structure (for example, B-Tree, Trie, m-way search tree, AVL tree, and Red-Black Tree in a nonlinear data structure) to increase the efficiency of operation while using resources of the computing device to a minimum. The above-described matter is just an example and the present disclosure is not limited thereto.

The data structure may include hyper-parameters of the neural network. In addition, the data structures, including the hyper-parameters of the neural network, may be stored in the computer readable medium. The hyper-parameter may be a variable which may be varied by the user. The hyper-parameter may include, for example, a learning rate, a cost function, the number of learning cycle iterations, weight initialization (for example, setting a range of weight values to be subjected to weight initialization), and Hidden Unit number (e.g., the number of hidden layers and the number of nodes in the hidden layer). The data structure is just an example and the present disclosure is not limited thereto.

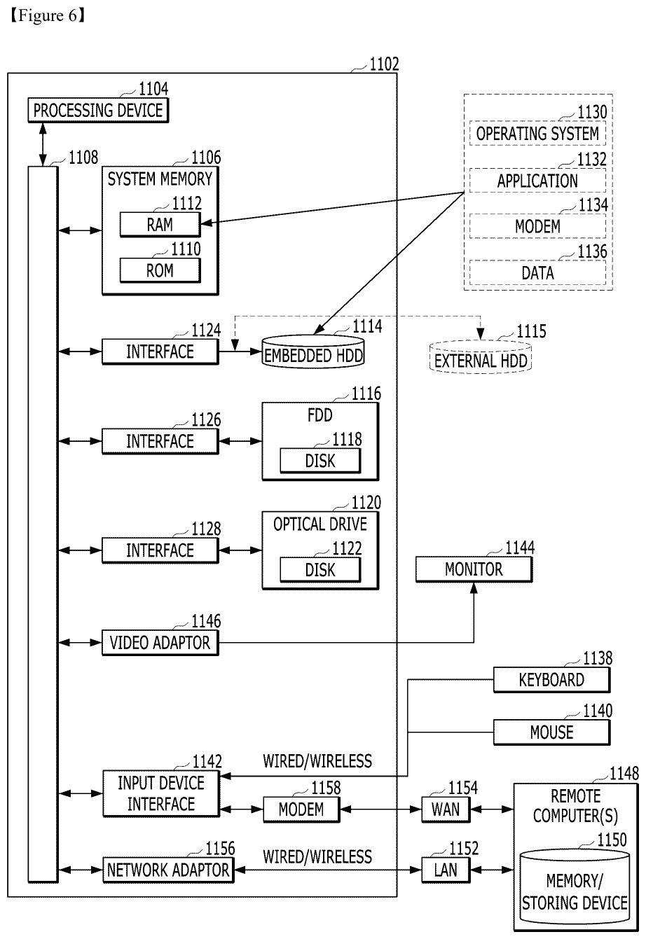

FIG. 6 is a normal and schematic view of an exemplary computing environment in which the exemplary embodiments of the present disclosure may be implemented.

It is described above that the present disclosure may be generally implemented by the computing device, but those skilled in the art will well know that the present disclosure may be implemented in association with a computer executable command which may be executed on one or more computers and/or in combination with other program modules and/or a combination of hardware and software.

In general, the program module includes a routine, a program, a component, a data structure, and the like that execute a specific task or implement a specific abstract data type. Further, it will be well appreciated by those skilled in the art that the method of the present disclosure can be implemented by other computer system configurations including a personal computer, a handheld computing device, microprocessor-based or programmable home appliances, and others (the respective devices may operate in connection with one or more associated devices as well as a single-processor or multi-processor computer system, a mini computer, and a main frame computer.

The exemplary embodiments described in the present disclosure may also be implemented in a distributed computing environment in which predetermined tasks are performed by remote processing devices connected through a communication network. In the distributed computing environment, the program module may be positioned in both local and remote memory storage devices.

The computer generally includes various computer readable media. Media accessible by the computer may be computer readable media regardless of types thereof and the computer readable media include volatile and non-volatile media, transitory and non-transitory media, and mobile and non-mobile media. As a non-limiting example, the computer readable media may include both computer readable storage media and computer readable transmission media. The computer readable storage media include volatile and non-volatile media, transitory and non-transitory media, and mobile and non-mobile media implemented by a predetermined method or technology for storing information such as a computer readable instruction, a data structure, a program module, or other data. The computer readable storage media include a RAM, a ROM, an EEPROM, a flash memory or other memory technologies, a CD-ROM, a digital video disk (DVD) or other optical disk storage devices, a magnetic cassette, a magnetic tape, a magnetic disk storage device or other magnetic storage devices or predetermined other media which may be accessed by the computer or may be used to store desired information, but are not limited thereto.

The computer readable transmission media generally implement the computer readable command, the data structure, the program module, or other data in a carrier wave or a modulated data signal such as other transport mechanism and include all information transfer media. The term “modulated data signal” means a signal acquired by setting or changing at least one of characteristics of the signal so as to encode information in the signal. As a non-limiting example, the computer readable transmission media include wired media such as a wired network or a direct-wired connection and wireless media such as acoustic, RF, infrared and other wireless media. A combination of any media among the aforementioned media is also included in a range of the computer readable transmission media.

An exemplary environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined processor among various commercial processors. A dual processor and other multi-processor architectures may also be used as the processing device 1104.

The system bus 1108 may be any one of several types of bus structures which may be additionally interconnected to a local bus using any one of a memory bus, a peripheral device bus, and various commercial bus architectures. The system memory 1106 includes a read only memory (ROM) 1110 and a random access memory (RAM) 1112. A basic input/output system (BIOS) is stored in the non-volatile memories 1110 including the ROM, the EPROM, the EEPROM, and the like and the BIOS includes a basic routine that assists in transmitting information among components in the computer 1102 at a time such as in-starting. The RAM 1112 may also include a high-speed RAM including a static RAM for caching data, and the like.

The computer 1102 also includes an interior hard disk drive (HDD) 1114 (for example, EIDE and SATA), in which the interior hard disk drive 1114 may also be configured for an exterior purpose in an appropriate chassis (not illustrated), a magnetic floppy disk drive (FDD) 1116 (for example, for reading from or writing in a mobile diskette 1118), and an optical disk drive 1120 (for example, for reading a CD-ROM disk 1122 or reading from or writing in other high-capacity optical media such as the DVD, and the like). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. An interface 1124 for implementing an exterior drive includes at least one of a universal serial bus (USB) and an IEEE 1394 interface technology or both of them.

The drives and the computer readable media associated therewith provide non-volatile storage of the data, the data structure, the computer executable instruction, and others. In the case of the computer 1102, the drives and the media correspond to storing of predetermined data in an appropriate digital format. In the description of the computer readable media, the mobile optical media such as the HDD, the mobile magnetic disk, and the CD or the DVD are mentioned, but it will be well appreciated by those skilled in the art that other types of media readable by the computer such as a zip drive, a magnetic cassette, a flash memory card, a cartridge, and others may also be used in an exemplary operating environment and further, the predetermined media may include computer executable commands for executing the methods of the present disclosure.

Multiple program modules including an operating system 1130, one or more application programs 1132, other program module 1134, and program data 1136 may be stored in the drive and the RAM 1112. All or some of the operating system, the application, the module, and/or the data may also be cached in the RAM 1112. It will be well appreciated that the present disclosure may be implemented in operating systems which are commercially usable or a combination of the operating systems.

A user may input instructions and information in the computer 1102 through one or more wired/wireless input devices, for example, pointing devices such as a keyboard 1138 and a mouse 1140. Other input devices (not illustrated) may include a microphone, an IR remote controller, a joystick, a game pad, a stylus pen, a touch screen, and others. These and other input devices are often connected to the processing device 1104 through an input device interface 1142 connected to the system bus 1108, but may be connected by other interfaces including a parallel port, an IEEE 1394 serial port, a game port, a USB port, an IR interface, and others.

A monitor 1144 or other types of display devices are also connected to the system bus 1108 through interfaces such as a video adapter 1146, and the like. In addition to the monitor 1144, the computer generally includes other peripheral output devices (not illustrated) such as a speaker, a printer, others.

The computer 1102 may operate in a networked environment by using a logical connection to one or more remote computers including remote computer(s) 1148 through wired and/or wireless communication. The remote computer(s) 1148 may be a workstation, a computing device computer, a router, a personal computer, a portable computer, a micro-processor based entertainment apparatus, a peer device, or other general network nodes and generally includes multiple components or all of the components described with respect to the computer 1102, but only a memory storage device 1150 is illustrated for brief description. The illustrated logical connection includes a wired/wireless connection to a local area network (LAN) 1152 and/or a larger network, for example, a wide area network (WAN) 1154. The LAN and WAN networking environments are general environments in offices and companies and facilitate an enterprise-wide computer network such as Intranet, and all of them may be connected to a worldwide computer network, for example, the Internet.

When the computer 1102 is used in the LAN networking environment, the computer 1102 is connected to a local network 1152 through a wired and/or wireless communication network interface or an adapter 1156. The adapter 1156 may facilitate the wired or wireless communication to the LAN 1152 and the LAN 1152 also includes a wireless access point installed therein in order to communicate with the wireless adapter 1156. When the computer 1102 is used in the WAN networking environment, the computer 1102 may include a modem 1158 or has other means that configure communication through the WAN 1154 such as connection to a communication computing device on the WAN 1154 or connection through the Internet. The modem 1158 which may be an internal or external and wired or wireless device is connected to the system bus 1108 through the serial port interface 1142. In the networked environment, the program modules described with respect to the computer 1102 or some thereof may be stored in the remote memory/storage device 1150. It will be well known that an illustrated network connection is exemplary and other means configuring a communication link among computers may be used.

The computer 1102 performs an operation of communicating with predetermined wireless devices or entities which are disposed and operated by the wireless communication, for example, the printer, a scanner, a desktop and/or a portable computer, a portable data assistant (PDA), a communication satellite, predetermined equipment or place associated with a wireless detectable tag, and a telephone. This at least includes wireless fidelity (Wi-Fi) and Bluetooth wireless technology. Accordingly, communication may be a predefined structure like the network in the related art or just ad hoc communication between at least two devices.

The wireless fidelity (Wi-Fi) enables connection to the Internet, and the like without a wired cable. The Wi-Fi is a wireless technology such as the device, for example, a cellular phone which enables the computer to transmit and receive data indoors or outdoors, that is, anywhere in a communication range of a base station. The Wi-Fi network uses a wireless technology called IEEE 802.11(a, b, g, and others) in order to provide safe, reliable, and high-speed wireless connection. The Wi-Fi may be used to connect the computers to each other or the Internet and the wired network (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or operate in a product including both bands (dual bands).

It will be appreciated by those skilled in the art that information and signals may be expressed by using various different predetermined technologies and techniques. For example, data, instructions, commands, information, signals, bits, symbols, and chips which may be referred in the above description may be expressed by voltages, currents, electromagnetic waves, magnetic fields or particles, optical fields or particles, or predetermined combinations thereof.

It may be appreciated by those skilled in the art that various exemplary logical blocks, modules, processors, means, circuits, and algorithm steps described in association with the exemplary embodiments disclosed herein may be implemented by electronic hardware, various types of programs or design codes (for easy description, herein, designated as software), or a combination of all of them. In order to clearly describe the intercompatibility of the hardware and the software, various exemplary components, blocks, modules, circuits, and steps have been generally described above in association with functions thereof. Whether the functions are implemented as the hardware or software depends on design restrictions given to a specific application and an entire system. Those skilled in the art of the present disclosure may implement functions described by various methods with respect to each specific application, but it should not be interpreted that the implementation determination departs from the scope of the present disclosure.

Various exemplary embodiments presented herein may be implemented as manufactured articles using a method, a device, or a standard programming and/or engineering technique. The term manufactured article includes a computer program, a carrier, or a medium which is accessible by a predetermined computer-readable storage device. For example, a computer-readable storage medium includes a magnetic storage device (for example, a hard disk, a floppy disk, a magnetic strip, or the like), an optical disk (for example, a CD, a DVD, or the like), a smart card, and a flash memory device (for example, an EEPROM, a card, a stick, a key drive, or the like), but is not limited thereto. Further, various storage media presented herein include one or more devices and/or other machine-readable media for storing information.

It will be appreciated that a specific order or a hierarchical structure of steps in the presented processes is one example of exemplary accesses. It will be appreciated that the specific order or the hierarchical structure of the steps in the processes within the scope of the present disclosure may be rearranged based on design priorities. Appended method claims provide elements of various steps in a sample order, but the method claims are not limited to the presented specific order or hierarchical structure.

The description of the presented exemplary embodiments is provided so that those skilled in the art of the present disclosure use or implement the present disclosure. Various modifications of the exemplary embodiments will be apparent to those skilled in the art and general principles defined herein can be applied to other exemplary embodiments without departing from the scope of the present disclosure. Therefore, the present disclosure is not limited to the exemplary embodiments presented herein, but should be interpreted within the widest range which is coherent with the principles and new features presented herein.

### MODE FOR INVENTION

Related contents in the best mode for carrying out the present disclosure are described as above.

1. A method for quantifying breast density or glandular tissue components using an ultrasound image performed by a computing device, the method comprising:
acquiring an ultrasound image;
identifying a breast region in the ultrasound image;
identifying a fibrous glandular tissue region in the ultrasound image; and
quantifying a density of a breast by analyzing the identified breast region and fibrous glandular tissue region.

2. The method of claim 1, wherein the identifying of the breast region in the ultrasound image comprises:
identifying the breast region by inputting the ultrasound image into a deep learning segmentation model.

3. The method of claim 2, wherein the identifying of the fibrous glandular tissue region in the ultrasound image comprises:
distinguishing and identifying the breast region and the fibrous glandular tissue region jointly by inputting the ultrasound image into the deep learning segmentation model.

4. The method of claim 1, wherein the quantifying of the density of the breast by analyzing the identified breast region and fibrous glandular tissue region comprises:
calculating a ratio of the fibrous glandular tissue region to a size of the identified breast region, and
quantifying a density of a breast tissue based on the calculated ratio.

5. The method of claim 4, wherein the quantifying of the density of the breast tissue comprises:
additionally quantifying the density of the breast tissue based on a mapping between the calculated ratio and a predefined breast density grade.

6. The method of claim 5, wherein the predefined breast density grade comprises an ACR BI-RADS Tissue Composition grade.

7. The method of claim 1, further comprising:
identifying a glandular tissue component region within the identified fibrous glandular tissue region.

8. The method of claim 7, wherein the identifying of the glandular tissue component region within the identified fibrous glandular tissue region comprises:
applying histogram equalization to the fibrous glandular tissue region, and
detecting the glandular tissue component region in the fibrous glandular tissue region based on a statistical threshold.

9. The method of claim 7, further comprising:
performing an operation of quantifying glandular tissue components by analyzing the identified fibrous glandular tissue region and the identified glandular tissue component region.

10. The method of claim 9, wherein the performing of the operation of quantifying the glandular tissue components by analyzing the identified fibrous glandular tissue region and the identified glandular tissue component region comprises:
calculating a ratio of a size of the glandular tissue component region to a size of the fibrous glandular tissue region, and
quantifying the glandular tissue components based on the calculated ratio.

11. The method of claim 10, wherein the quantifying of the glandular tissue components comprises:
additionally quantifying the glandular tissue components based on a mapping between the calculated ratio and a predefined glandular tissue component grade.

12. The method of claim 11, wherein the predefined glandular tissue component grade comprises a Glandular Tissue Composition (GTC) grade.

13. A computer program stored in a non-transitory computer-readable storage medium, wherein when the computer program is executed by one or more processors, the computer program allows the one or more processors to perform following operations for quantifying breast density and glandular tissue components using an ultrasound image, the operations comprising:
an operation of acquiring an ultrasound image;
an operation of identifying a breast region in the ultrasound image;
an operation of identifying a fibrous glandular tissue region in the ultrasound image; and
an operation of quantifying a density of a breast by analyzing the identified breast region and fibrous glandular tissue region.

14. The computer program of claim 13, wherein the operation of identifying the breast region in the ultrasound image comprises:
an operation of identifying the breast region by inputting the ultrasound image into a deep learning segmentation model.

15. The computer program of claim 14, wherein the operation of identifying the fibrous glandular tissue region in the ultrasound image comprises:
an operation of distinguishing and identifying the breast region and the fibrous glandular tissue region jointly by inputting the ultrasound image into the deep learning segmentation model.

16. The computer program of claim 13, wherein the operation of quantifying the density of the breast by analyzing the identified breast region and fibrous glandular tissue region comprises:
an operation of calculating a ratio of the fibrous glandular tissue region to a size of the identified breast region, and
an operation of quantifying a density of a breast tissue based on the calculated ratio.

17. (canceled)

18. (canceled)

19. (canceled)

20. (canceled)

21. (canceled)

22. A computing device comprising:
at least one processor; and
a memory,
wherein the at least one processor is configured to:
acquire an ultrasound image,
identify a breast region in the ultrasound image,
identify a fibrous glandular tissue region in the ultrasound image, and
quantify a density of a breast by analyzing the identified breast region and fibrous glandular tissue region.

23. The computing device of claim 22, wherein the at least one processor is configured to identify the breast region by inputting the ultrasound image into a deep learning segmentation model.

24. The computing device of claim 23, wherein the at least one processor is configured to distinguish and identify the breast region and the fibrous glandular tissue region jointly by inputting the ultrasound image into the deep learning segmentation model.

25. The computing device of claim 22, wherein the at least one processor is configured to:
calculate a ratio of the fibrous glandular tissue region to a size of the identified breast region, and
quantify a density of a breast tissue based on the calculated ratio.

26. (canceled)

27. (canceled)

28. (canceled)
